(19) United States
(12) Patent Application Publication
(10) Pub. No.: US 2008/0113974 A1
(43) Pub. Date: May 15, 2008

(54) Heterocyclic Oxime Compounds, A Process For Their Preparation And Pharmaceutical Compositions Containing Them.

(75) Inventors: Aurelie Hurtevent, Lille (FR); Jean-Martial L' Helgoual' Ch, Lille (FR); Pascal Carato, Lille (FR); Nicolas Lebegue, La Bassee (FR); Veronique Leclerc, Avelin (FR); Pascal Berthelot, Haubourdin (FR); Catherine Dacquet, Villeneuve D'Ascq (FR); Alain Ktorza, Nogent Sur Marne (FR); Daniel-Henri Caignard, Boisemont (FR)

Correspondence Address: THE FIRM OF HUESCHEN AND SAGE, SEVENTH FLOOR, KALAMAZOO BUILDING, 107 WEST MICHIGAN AVENUE, KALAMAZOO, MI, 49007, US

(73) Assignee: LES LABORATOIRES SERVIER, COURBRVOIE CEDEX (FR)

(21) Appl. No.: 11/883,121

(22) PCT Filed: Jan. 26, 2006
(86) PCT No.: PCT/FR06/00175

§ 371 Date: Jul. 26, 2007

(30) Foreign Application Priority Data
Jan. 27, 2005 (FR) 05.00841

(51) Int. Cl. C07D 279/16; C07D 265/36; A61K 31/5415; A61K 31/538

(52) U.S. Cl. 514/224.2; 544/105; 544/52; 514/230.5

(57) ABSTRACT

Compounds of formula (I):

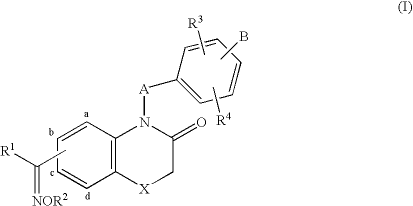

wherein:
X represents an oxygen atom or a sulphur atom,
R¹, R², R³ and R⁴ are as defined in the description,
A represents an alkylene chain as defined in the description, and
B represents an alkyl or alkenyl group substituted by a group

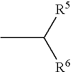

or R⁷, or B represents a group

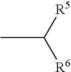

or R⁷.

The present invention relates to new heterocyclic oxime compounds, to a process for their preparation and to pharmaceutical compositions containing them.

The compounds described in the present invention are new and have pharmacological properties that are of special interest: they are excellent hypoglycaemic and hypolipaemic agents.

The treatment of non-insulin-dependent type II diabetes remains unsatisfactory despite the introduction onto the market of a large number of oral hypoglycaemic compounds intended to facilitate the secretion of insulin and to promote its action in peripheral target tissues.

During the last ten years, a class of compounds having a thiazolidinedione structure (U.S. Pat. No. 5,089,514, U.S. Pat. No. 5,306,726) has demonstrated a marked anti-diabetic activity by promoting sensitivity to insulin in target peripheral tissues (skeletal muscle, liver, adipose tissue) of animal models having non-insulin-dependent type II diabetes. Those compounds also lower the levels of insulin and of lipids in the same animal models and induce in vitro differentiation of preadipocyte cell lines into adipocyte cell lines (A. Hiragun et al., J. Cell. Physiol., 1988, 134, 124-130; R. F. Kleitzen et al., Mol. Pharmacol., 1992, 41, 393-398). The treatment of preadipocyte cell lines with the thiazolidinedione rosiglitazone brings about inducement of the expression of specific genes of the lipid metabolism, such as aP2 and adipsin, and also the expression of the glucose transporters GLUT1 and GLUT4, suggesting that the effect of the thiazolidinediones observed in vivo may be mediated via adipose tissue. That specific effect is obtained by the stimulation of nuclear transcription factors: <<peroxisome proliferator-activated receptor gamma>> (PPAR γ2). Such compounds are capable of restoring sensitivity to insulin in peripheral tissues, such as adipose tissue or skeletal muscle (J. E. Gerich, New Engl. Med., 19, 321, 1231-1245).

Compounds having a thiazolidinedione structure (troglitazone, rosiglitazone) have demonstrated disturbing side effects in man, however, especially liver problems (Script N^{o} 2470, 1999, Sep. 8, 25).

A large number of hypoglycaemic agents have significant side effects (hepatic, cardiac, haematopoietic), which limit their long-term use in the treatment of non-insulin-dependent type II diabetes.

The development of new therapeutic agents that are less toxic and that are active over the long term is absolutely necessary in this pathology.

Moreover, hyperlipidaemia is often observed in diabetics (Diabetes Care, 1995, 18 (supplement 1), 86/8/93). The association of hyperglycaemia with hyperlipidaemia increases the risk of cardiovascular disease in diabetics. Hyperglycaemia, hyperlipidaemia and obesity have become pathologies of the modern world marked by the intake of food in large quantities and a chronic lack of exercise.

The increase in frequency of those pathologies calls for the development of new therapeutic agents that are active in such disorders: compounds having an excellent hypoglycaemic and hypolipaemic activity whilst avoiding the side effects observed with thiazolidinediones are consequently very beneficial in the treatment and/or prophylaxis of those pathologies, and are indicated especially in the treatment of non-insulin-dependent type II diabetes for reducing peripheral insulin resistance and for normalising glucose control.

In addition to the fact that they are new, the compounds of the present invention meet the above pharmacological criteria and are excellent hypoglycaemic and hypolipaemic agents.

The present invention relates more especially to the compounds of formula (I):

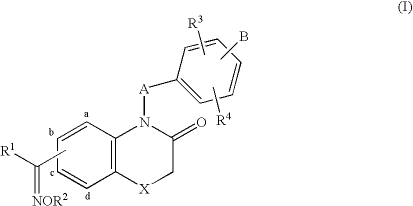

wherein:
X represents an oxygen atom or a sulphur atom,
A represents a (C₁-C₆)alkylene chain in which a CH₂ group may optionally be replaced by a hetero atom selected from oxygen and sulphur, or by an NRₐ group (wherein Rₐ represents a hydrogen atom or a linear or branched (C₁-C₆)alkyl group), or by a phenylene or naphthylene group, R¹ and R², which may be identical or different, each represents a hydrogen atom, a linear or branched (C₁-C₆)alkyl group, a linear or branched (C₂-C₆)alkenyl group, a linear or branched (C₂-C₆)alkynyl group, an aryl group, an aryl-(C₁-C₆)alkyl group in which the alkyl moiety may be linear or branched, an aryl-(C₂-C₆)alkenyl group in which the alkenyl moiety may be linear or branched, an aryl-(C₂-C₆)alkynyl group in which the alkynyl moiety may be linear or branched, a heteroaryl group, a heteroaryl-(C₁-C₆)alkyl group in which the alkyl moiety may be linear or branched, a heteroaryl-(C₂-C₆)alkenyl group in which the alkenyl moiety may be linear or branched, a heteroaryl-(C₂-C₆)alkynyl group in which the alkynyl moiety may be linear or branched, a (C₃-C₈)cycloalkyl group, a (C₃-C₈)cycloalkyl-(C₁-C₆)alkyl group in which the alkyl moiety may be linear or branched or a linear or branched (C₁-C₆)polyhaloalkyl group,
R³ and R⁴, which may be identical or different, each represents a hydrogen atom, a halogen atom, or an R, OR or NRR′ group wherein R and R′, which may be identical or different, each represents a hydrogen atom or a linear or branched (C₁-C₆)alkyl group, a linear or branched (C₂-C₆)alkenyl group, a linear or branched (C₂-C₆)alkynyl group, an aryl group, an aryl-(C₁-C₆)alkyl group in which the alkyl moiety may be linear or branched, an aryl-(C₂-C₆)alkenyl group in which the alkenyl moiety may be linear or branched, an aryl-(C₂-C₆)alkynyl group in which the alkynyl moiety may be linear or branched, a heteroaryl group, a heteroaryl-(C₁-C₆)alkyl group in which the alkyl moiety may be linear or branched, a heteroaryl-(C₂-C₆)alkenyl group in which the alkenyl moiety may be linear or branched, a heteroaryl-(C₂-C₆)alkynyl group in which the alkynyl moiety may be linear or branched, a (C₃-C₈)cycloalkyl group, a (C₃-C₈)cycloalkyl-(C₁-C₆)alkyl group in which the alkyl moiety may be linear or branched or a linear or branched (C₁-C₆)polyhaloalkyl group,
   or R³ and R⁴, together with the carbon atoms carrying them, when they are carried by two adjacent carbon atoms, form a ring that comprises 5 or 6 ring members and that may optionally contain a hetero atom selected from oxygen, sulphur and nitrogen,
B represents a linear or branched (C₁-C₆)alkyl group or a linear or branched (C₂-C₆)alkenyl group, those groups being substituted:
   by a group of formula (II):

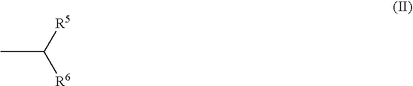

wherein:

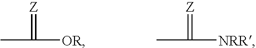

R⁵ represents a

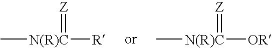

group wherein Z represents an oxygen atom or a sulphur atom and R and R′, which may be identical or different, are as defined hereinbefore,
and R⁶ represents an aryl group, an arylalkyl group in which the alkyl moiety contains from 1 to 6 carbon atoms and may be linear or branched, a heteroaryl group, a heteroarylalkyl group in which the alkyl moiety contains from 1 to 6 carbon atoms and may be linear or branched, CN, tetrazole

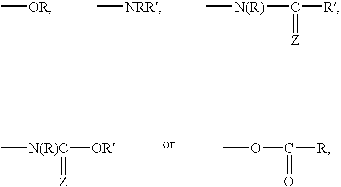

wherein Z is as defined hereinbefore and R and R′, which may be identical or different, may take the same meanings as defined hereinbefore,
or by a group R⁷, R⁷ representing a CN, tetrazole,

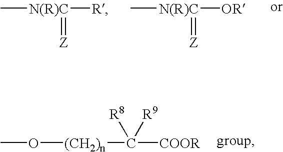

wherein Z is as defined hereinbefore and R and R′, which may be identical or different, may take the same meanings as defined hereinbefore, n represents 0, 1, 2, 3, 4, 5 or 6, and R⁸ and R⁹, which may be identical or different, each represents a hydrogen atom or a linear or branched (C₁-C₆)alkyl group, wherein R⁸ and R⁹ cannot simultaneously represent a hydrogen atom,
or B represents a group of formula (II) or a group R⁷ as defined hereinbefore,
   it being understood that:
the oxime R¹—C(═N—OR²)— may have the Z or E configuration,
aryl is understood to mean a phenyl, naphthyl or biphenyl group, wherein those groups may optionally be partially hydrogenated,
heteroaryl is understood to mean any aromatic mono- or bi-cyclic group containing from 5 to 10 ring members, which in the case of bicyclic heteroaryl groups may optionally be partially hydrogenated on one of the rings, and containing from 1 to 3 hetero atoms selected from oxygen, nitrogen and sulphur,
   wherein the aryl and heteroaryl groups so defined may optionally be substituted by from 1 to 3 identical or different groups selected from linear or branched (C₁-C₆)alkyl, linear or branched (C₁-C₆)polyhaloalkyl, linear or branched (C₁-C₆)alkoxy, hydroxy, carboxy, formyl, linear or branched (C₁-C₆)acyl, aroyl, NR_{b}R_{c} (wherein R_{b} and R_{c}, which may be identical or different, each represents a hydrogen atom, a linear or branched (C₁-C₆)alkyl group, an aryl group or a heteroaryl group), ester, amido, nitro, cyano, and halogen atoms,
   to their enantiomers and diastereoisomers, and also to pharmaceutically acceptable addition salts thereof with an acid or a base.

Amongst the pharmaceutically acceptable acids there may be mentioned, without implying any limitation, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, methanesulphonic acid, camphoric acid, oxalic acid, etc. . . . .

Amongst the pharmaceutically acceptable bases there may be mentioned, without implying any limitation, sodium hydroxide, potassium hydroxide, triethylamine, tert-butylamine, etc.

Preferably, the group R¹—C(═N—OR²)— is in the b or c position.

A preferred group for R³ and R⁴ is the hydrogen atom.

Preferably, A represents an alkylene chain in which a CH₂ group may optionally be replaced by a hetero atom and more especially by an oxygen atom.

The invention relates more especially to compounds of formula (I) wherein A represents an ethyleneoxy group.

Preferred R² groups are the hydrogen atom and alkyl groups, such as, for example, the methyl group.

R¹ advantageously represents a phenyl group that is unsubstituted or substituted by one or more substituents selected from groups such as alkyl, alkoxy, and halogen atoms, and R¹ represents more especially an unsubstituted phenyl group.

Preferred groups B are alkyl or alkenyl groups, and more especially alkyl groups, substituted by

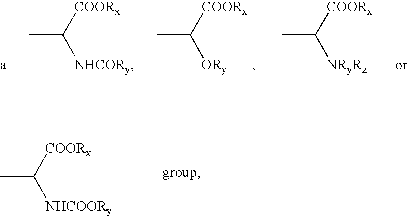

wherein Rₓ, R_{y} and R_{z}, which may be identical or different, each represents:
a hydrogen atom or an alkyl group, such as, for example, a methyl, ethyl, propyl, isopropyl, butyl, isobutyl, tert-butyl, pentyl, isopentyl, neopentyl or hexyl group, a polyhaloalkyl group, such as, for example, a trifluoromethyl or trifluoroethyl group, or a phenyl or benzyl group.

The invention relates more especially to compounds of formula (I) wherein B represents an alkyl or alkenyl group substituted by a

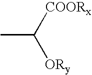

group wherein Rₓ and R_{y} are are as defined hereinbefore.

Likewise advantageously, B represents a group

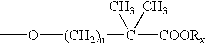

wherein n and Rₓ are as defined hereinbefore.

Even more especially, B represents a group

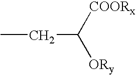

wherein Rₓ and R_{y} are as defined hereinbefore.

The invention relates very advantageously to compounds of formula (I) wherein:

### X represents an oxygen atom or a sulphur atom,

### A represents a

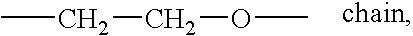

### R³ and R⁴ simultaneously represent a hydrogen atom,

### R² represents a hydrogen atom or an alkyl group,

### R¹ represents an unsubstituted phenyl group, and

### B represents a group

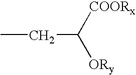

wherein Rₓ and R_{y} are as defined hereinbefore.

Even more especially, the invention relates to the following compounds of formula (I):
ethyl 2-ethoxy-3-[4-(2-{7-[(methoxyimino)(phenyl)methyl]-3-oxo-2,3-dihydro-4H-1,4-benzothiazin-4-yl}ethoxy)phenyl]propanoate,
ethyl 3-[4-(2-{7-[(methoxyimino)(phenyl)methyl]-3-oxo-2,3-dihydro-4H-1,4-benzothiazin-4-yl}ethoxy)phenyl]-2-(2,2,2-trifluoroethoxy)propanoate,
ethyl 2-ethoxy-3-[4-(2-{6-[(methoxyimino)(phenyl)methyl]-3-oxo-2,3-dihydro-4H-1,4-benzoxazin-4-yl}ethoxy)phenyl]propanoate,
ethyl 2-ethoxy-3-[4-(2-{7-[(hydroxyimino)(phenyl)methyl]-3-oxo-2,3-dihydro-4H-1,4-benzothiazin-4-yl}ethoxy)phenyl]propanoate,
ethyl 3-[4-(2-{7-[(hydroxyimino)(phenyl)methyl]-3-oxo-2,3-dihydro-4H-1,4-benzothiazin-4-yl}ethoxy)phenyl]-2-(2,2,2-trifluoroethoxy)propanoate
ethyl 2-ethoxy-3-[4-(2-{6-[(hydroxyimino)(phenyl)methyl]-3-oxo-2,3-dihydro-4H-1,4-benzoxazin-4-yl}ethoxy)phenyl]propanoate,
2-ethoxy-3-[4-(2-{7-[(hydroxyimino)(phenyl)methyl]-3-oxo-2,3-dihydro-4H-1,4-benzothiazin-4-yl}ethoxy)phenyl]propanoic acid,
2-ethoxy-3-[4-(2-{7-[(methoxyimino)(phenyl)methyl]-3-oxo-2,3-dihydro-4H-1,4-benzothiazin-4-yl}ethoxy)phenyl]propanoic acid,
3-[4-(2-{7-[(hydroxyimino)(phenyl)methyl]-3-oxo-2,3-dihydro-4H-1,4-benzothiazin-4-yl}ethoxy)phenyl]-2-(2,2,2-trifluoroethoxy)propanoic acid,
3-[4-(2-{7-[(methoxyimino)(phenyl)methyl]-3-oxo-2,3-dihydro-4H-1,4-benzothiazin-4-yl}ethoxy)phenyl]-2-(2,2,2-trifluoroethoxy)propanoic acid,
2-ethoxy-3-[4-(2-{6-[(hydroxyimino)(phenyl)methyl]-3-oxo-2,3-dihydro-4H-1,4-benzoxazin-4-yl}ethoxy)phenyl]propanoic acid,
2-ethoxy-3-[4-(2-{6-[(methoxyimino)(phenyl)methyl]-3-oxo-2,3-dihydro-4H-1,4-benzoxazin-4-yl}ethoxy)phenyl]propanoic acid.

The enantiomers, diastereoisomers, and also pharmaceutically acceptable addition salts with an acid or a base of the preferred compounds of the invention form an integral part of the invention.

The present invention relates also to a process for the preparation of compounds of formula (I) which is characterised in that there is used as starting material a compound of formula (III):

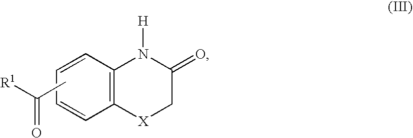

wherein R¹ and X are as defined for formula (I), which is condensed in basic medium with a compound of formula (IV):

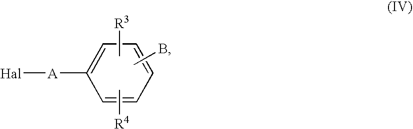

wherein A, B, R³ and R⁴ are as defined for formula (I) and Hal represents a halogen atom, to yield a compound of formula (V):

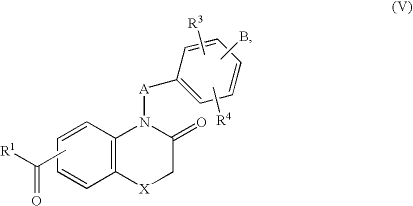

wherein R¹, R³, R⁴, A, B and X are as defined for formula (I),
which is subjected to the action of a compound of formula R²O—NH₂ wherein R² is as defined for formula (I) to yield a compound of formula (I):

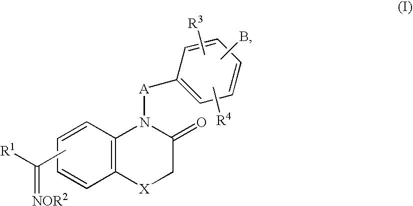

which may be purified according to a conventional separation technique, is converted, if desired, into addition salts with a pharmaceutically acceptable acid or base, and is optionally separated into isomers according to a conventional separation technique.

An advantageous variant relates to a process for the preparation of the compounds of formula (I) which is characterised in that there is used as starting material a compound of formula (III):

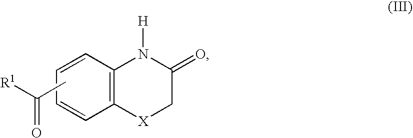

wherein R¹ and X are as defined for formula (I),
which is condensed with a compound of formula R²O—NH₂ wherein R² is as defined for formula (I) to yield a compound of formula (VI):

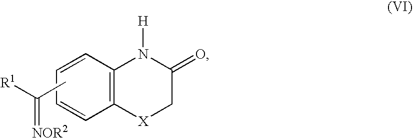

wherein R¹, R² and X are as defined for formula (I),
which is condensed in basic medium with a compound of formula (IV):

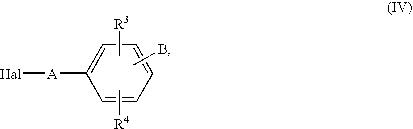

wherein A, B, R³ and R⁴ are as defined for formula (I) and Hal represents a halogen atom, to yield a compound of formula (I):

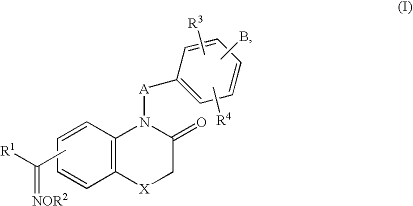

which may be purified according to a conventional separation technique, is converted, if desired, into addition salts with a pharmaceutically acceptable acid or base, and is optionally separated into isomers according to a conventional separation technique.

The compounds of formula (III) are commercial products, or readily obtainable by the person skilled in the art by conventional chemical reactions, or described in the literature.

The compounds of the present invention have very valuable pharmacological properties.

The compounds demonstrate especially an excellent activity in lowering blood glucose levels. As a result of such properties they can be used therapeutically in the treatment and/or prophylaxis of hyperglycaemia, dyslipidaemia and, more especially, in the treatment of non-insulin-dependent type II diabetes, glucose intolerance, disorders associated with syndrome X (including hypertension, obesity, insulin resistance, atherosclerosis, hyperlipidaemia), coronary artery disease and other cardiovascular diseases (including arterial hypertension, cardiac insufficiency, venous insufficiency), renal disorders (including glomerulonephritis, glomerulosclerosis, nephrotic syndrome, hypertensive nephrosclerosis), retinopathy, disorders associated with the activation of endothelial cells, psoriasis, polycystic ovary syndrome, dementia, diabetic complications and osteoporosis.

They can be used as aldose reductase inhibitors for improving cognitive functions in dementia and for the complications of diabetes, intestinal inflammatory disorders, myotonic dystrophy, pancreatitis, arteriosclerosis, xanthoma.

The activity of these compounds is also recommended for the treatment and/or prophylaxis of other diseases, including type I diabetes, hypertriglyceridaemia, syndrome X, insulin resistance, dyslipidaemia in diabetics, hyperlipidaemia, hypercholesterolaemia, arterial hypertension, cardiac insufficiency, and cardiovascular disease, especially atherosclerosis.

The compounds are furthermore indicated for use in the regulation of appetite, especially in the regulation of food intake in subjects suffering from disorders such as obesity, anorexia, bulimia and anorexia nervosa.

The compounds can accordingly be used in the prevention or treatment of hypercholesterolaemia, obesity with advantageous effects on hyperlipidaemia, hyperglycaemia, osteoporosis, glucose intolerance, insulin resistance or disorders in which insulin resistance is a secondary physiopathological mechanism.

The use of those compounds enables reduction of total cholesterol, body weight, leptin resistance, plasma glucose, triglycerides, LDLs, VLDLs and also plasma free fatty acids. The compounds can be used in association with HMG CoA reductase inhibitors, fibrates, nicotinic acid, cholestyramine, colestipol, probucol, GLP1, metformin, biguanides or glucose reabsorption inhibitors and can be administered together or at different times to act in synergy in the patient treated.

They furthermore exhibit activity in cancer pathologies and especially hormone-dependent cancers, such as breast cancer and colon cancer, and also have an inhibiting effect on the angiogenesis processes implicated in those pathologies.

Amongst the pharmaceutical compositions according to the invention there may mentioned more especially those which are suitable for oral, parenteral, nasal, per- or trans-cutaneous, rectal, perlingual, ocular or respiratory administration and especially tablets or dragées, sublingual tablets, sachets, paquets, gelatin capsules, glossettes, lozenges, suppositories, creams, ointments, dermal gels and drinkable or injectable ampoules.

The dosage varies in accordance with the sex, age and weight of the patient, the administration route, the nature of the therapeutic indication or of any associated treatments and ranges from 0.1 mg to 1 g per 24 hours taken in 1 or more administrations.

The present invention relates also to a new association between a heterocyclic compound of formula (I) as defined hereinbefore and an antioxidant agent for obtaining pharmaceutical compositions for use in the treatment and/or prevention of obesity and overweight characterised by a body mass index greater than 25.

The antioxidant agents according to the invention are, more especially, anti-free radical agents or free-radical trapping agents, antilipoperoxidant agents, chelating agents or agents capable of regenerating endogenous antioxidants such as glutathione, vitamin C or vitamin E, and also addition salts thereof with a pharmaceutically acceptable acid or base.

The antioxidant agent of the association according to the invention is more preferably represented by quinone compounds such as ubiquinone or coenzyme Q₁₀, which acts as a free-radical trapping agent but which is also capable of regenerating vitamin E.

A preferred association according to the invention is 3-{4-[6-(hydroxyiminophenyl)methyl-3-oxo-4H-benzo[1,4]oxazin-3-yl)ethoxy]phenyl}-2-ethoxypropanoic acid and coenzyme Q₁₀.

Furthermore, the association according to the invention has entirely surprising pharmacological properties: the Applicant has, in fact, demonstrated that a synergy exists between the two compounds of the association allowing a very significant reduction in body fat to be obtained, making it useful in the treatment and/or prevention of obesity and of overweight characterised by a body mass index greater than 25.

In the United States, obesity affects 20% of men and 25% of women. Patients having a body mass index (BMI=weight (kg)/height² (m²)) greater than or equal to 30 are considered to be obese (Int. J. Obes., 1998, 22, 39-47; Obesity Lancet, 1997, 350, 423-426). Obesity (BMI≧30) and overweight (25<BMI<30) can have various origins: they may come about following deregulation of food intake, following hormonal disturbance, or following administration of a treatment: treating type II diabetes with sulphonylureas causes patients to gain weight. Similarly, in type I (insulin-dependent) diabetes, insulin therapy is also a cause of weight gain in patients (In Progress in Obesity Research, 8^{th} International Congress on Obesity, 1999, 739-746; Annals of Internal Medicine, 1998, 128, 165-175).

Obesity and overweight are well-established risk factors for cardiovascular diseases: they are associated with a significant increase in the risk of cerebro-vascular accidents and non-insulin-dependent diabetes, because they predispose to insulin resistance, to dyslipidaemia and to the appearance of macrovascular disorders (nephropathy, retinopathy, angiopathy). Further pathologies are the consequence of obesity or overweight: there may be mentioned, in particular, vesicular calculi, respiratory dysfunction, several forms of cancer and, in the case of very severe obesity, premature death (N. Engl. J. Med., 1995, 333, 677-385; JAMA, 1993, 270, 2207-2212).

The association according to the invention allows a weight loss to be obtained which, even if moderate, significantly reduces all the risk factors associated with obesity (Int. J. Obes., 1997, 21, 55-9; Int. J. Obes., 1992, 21, S5-9).

The association according to the invention will therefore be found to be useful in the treatment and/or prevention of obesity and of overweight characterised by a body mass index greater than 25.

The invention accordingly relates to the use of the association between a compound of formula (I) and an antioxidant agent in obtaining pharmaceutical compositions intended for the treatment and/or prevention of obesity and of overweight characterised by a body mass index greater than 25 and less than 30.

In particular, the association according to the invention is useful in the treatment and/or prevention of obesity and of overweight characterised by a body mass index greater than 25 and less than 30 caused by a therapeutic treatment, such as treatment for type I or type II diabetes.

The invention accordingly relates to the use of the association between a compound of formula (I) and an antioxidant agent in obtaining pharmaceutical compositions intended for the treatment and/or prevention of obesity and of overweight characterised by a body mass index greater than 25 and less than 30 caused by a therapeutic treatment, such as treatment for type I or type II diabetes.

The invention relates also to pharmaceutical compositions comprising the association between a compound of formula (I) and an antioxidant agent, as defined hereinbefore, in combination with one or more pharmaceutically acceptable excipients.

Among the pharmaceutical compositions according to the invention there may be mentioned, more especially, those that are suitable for oral, parenteral or nasal administration, tablets or dragées, sublingual tablets, gelatin capsules, lozenges, suppositories, creams, ointments, dermal gels, etc.

In particular, the invention relates to pharmaceutical compositions comprising a compound of formula (I) as defined hereinbefore and an antioxidant agent, such as coenzyme Q₁₀ or vitamin E, in combination with one or more pharmaceutically acceptable excipients.

The dosage used varies according to the sex, age and weight of the patient, the administration route, the nature of the therapeutic indication or of any associated treatments and ranges from 0.1 mg to 1 g of each component of the association per 24 hours in one or more administrations.

The Preparations and Examples which follow illustrate the invention but do not limit it in any way.

### Preparation 1: 7-{([Methoxyimino]phenylmethyl}-4H-benzo[1,4]thiazin-3-one

### Step A: 7-Benzoyl-4H-benzo[1,4]thiazin-3-one

7.4 g of benzoic acid and then 10 g of 4H-benzo[1,4]thiazin-3-one are added to 100 g of polyphosphoric acid. The mixture is heated at 160° C. for 6 hours with mechanical stirring.

The solution is then hydrolysed, and the precipitate is suction-filtered off and washed with a saturated sodium hydrogen carbonate solution and then with absolute alcohol. The solid obtained is recrystallised from toluene to yield the title product in the form of a beige powder.

Melting point: 211-213° C.

### Step B: 7-{[Methoxyimino]phenylmethyl}-4H-benzo[1,4]thiazin-3-one

There are dissolved in 40 ml of methanol 5 g of the compound obtained in Step A and then 3 g of methoxylamine hydrochloride and 5.7 g of pyridine. The mixture is heated at reflux for 4 hours with stirring. After heating is ceased, the product is left to precipitate. The precipitate is filtered off and corresponds to the “E” isomer of the title compound. The filtrate is hydrolysed and acidified using 3N HCl, and then the precipitate, which corresponds to the “Z” isomer of the title compound, is filtered off.

Melting point: 214-216° C. (“E” isomer)
138-140° C. (mixture of “E” and “Z”. 72/28%)

### Preparation 2: 6-{[Methoxyimino]phenylmethyl}-4H-benzo[1,4]oxazin-3-one

### Step A: 6-Benzoyl-4H-benzo[1,4]oxazin-3-one

8.2 g of benzoic acid and then 10 g of 2H-[1,4]-benzoxazin-3-one are added to 100 g of polyphosphoric acid. The mixture is heated at 160° C. for 6 hours with mechanical stirring. The solution is then hydrolysed, and the precipitate is suction-filtered off and washed with a saturated sodium hydrogen carbonate solution and then with absolute alcohol. The solid obtained is recrystallised from toluene to yield the title product in the form of a white powder.

Melting point: 154-156° C.

### Step B: 6-{[Methoxyimino]phenylmethyl}-4H-benzo[1,4]oxazin-3-one

There are dissolved in 40 ml of methanol 5 g of the compound obtained in Step A and then 3 g of methoxylamine hydrochloride and 5.7 g of pyridine. The mixture is heated at reflux for 4 hours with stirring. After heating is ceased, the filtrate is hydrolysed and acidified using 3N HCl and then the precipitate, which corresponds to the title compound, is filtered off.

Melting point: 147-149° C.

### Preparation 3: Ethyl 3-{4-[2-(7-benzoyl-3-oxo-4H-benzo[1,4]thiazin-4-yl)ethoxy]phenyl}-2-ethoxypropanoate

1 g of the compound obtained in Step A of Preparation 1 is dissolved in 30 ml of dimethylformamide and then 1 g of potassium carbonate is added and stirring is carried out at 80° C. for one hour. Ethyl 3-[4-(2-chloroethoxy)phenyl]-2-ethoxypropanoate (1.67 g) is dissolved in a minimum amount of dimethylformamide and stirring is carried out overnight at 110° C. The dimethylformamide is then evaporated off, and the solution is subsequently hydrolysed and acidified. The aqueous phase is extracted with dichloromethane, dried over magnesium sulphate and filtered and the organic phase is evaporated. The residue is purified on silica gel using toluene and ethyl acetate (8/2) as eluant to yield the title product in the form of an oil.

### Preparation 4: Ethyl 3-{4-[2-(7-benzoyl-3-oxo-4H-benzo[1,4]thiazin-4-yl)ethoxy]phenyl}-2-(2,2,2-trifluoroethoxy)propanoate

1 g of the compound obtained in Step A of Preparation 1 is dissolved in 30 ml of dimethylformamide and then 1 g of potassium carbonate is added and stirring is carried out at 80° C. for one hour. Ethyl 3-[4-(2-chloroethoxy)phenyl]-2-(2,2,2-trifluoroethoxy)propanoate (1.97 g) is dissolved in a minimum amount of dimethylformamide and stirring is carried out overnight at 110° C. The dimethylformamide is then evaporated off, and the solution is subsequently hydrolysed and acidified. The aqueous phase is extracted with dichloromethane, dried over magnesium sulphate and filtered and the organic phase is evaporated. The residue is purified on silica gel using toluene and ethyl acetate (8/2) as eluant to yield the title product in the form of an oil.

### Preparation 5: Ethyl 3-{4-[2-(6-benzoyl-3-oxo-4H-benzo[1,4]oxazin-4-yl)ethoxy]phenyl}-2-ethoxypropanoate

1 g of the compound obtained in Step A of Preparation 2 is dissolved in 30 ml of dimethylformamide and then 1.1 g of potassium carbonate is added and stirring is carried out at 80° C. for one hour. Ethyl 3-[4-(2-chloroethoxy)phenyl]-2-ethoxypropanoate (1.78 g) is dissolved in a minimum amount of dimethylformamide and stirring is carried out overnight at 110° C.

The dimethylformamide is then evaporated off, and the solution is subsequently hydrolysed and acidified. The aqueous phase is extracted with dichloromethane, dried over magnesium sulphate and filtered and the organic phase is evaporated. The residue is purified on silica gel using toluene and ethyl acetate (8/2) as eluant to yield the title product in the form of an oil.

### Preparation 6: 6-{[Methoxyimino]phenylmethyl}-4H-benzo[1,4]thiazin-3-one

### Step A: 6-Benzoyl-4H-benzo[1,4]thiazin-3-one

### Step B: 6-{[Methoxyimino]phenylmethyl}-4H-benzo[1,4]thiazin-3-one

The procedure is as in Preparation 2 with the replacement of 2H-[1,4]-benzoxazin-3-one with 2H-[1,4]-benzothiazin-3-one.

### Preparation 7: 7-{[Methoxyimino]phenylmethyl}-4H-benzo[1,4]oxazin-3-one

### Step A: 7-Benzoyl-4H-benzo[1,4]oxazin-3-one

### Step B: 7-{[Methoxyimino]phenylmethyl}-4H-benzo[1,4]oxazin-3-one

The procedure is as in Preparation 1 with the replacement of 4H-benzo[1,4]thiazin-3-one with 4H-[1,4]-benzoxazin-3-one.

### Preparation 8: Ethyl 3-{4-[2-(7-benzoyl-3-oxo-4H-benzo[1,4]oxazin-4-yl)ethoxy]phenyl}-2-ethoxypropanoate

The procedure is as in Preparation 5 with the replacement of the product obtained in Step A of Preparation 2 with the product obtained in Step A of Preparation 6.

### Preparation 9: Ethyl 3-{4-[2-(7-benzoyl-3-oxo-4H-benzo[1,4]thiazin-4-yl)ethoxy]phenyl}-2-(2,2,2-trifluoroethoxy)propanoate

The procedure is as in Preparation 4 with the replacement of the product obtained in Step A of Preparation 1 with the product obtained in Step A of Preparation 6.

### Preparation 10: Ethyl 3-{4-[2-(7-benzoyl-3-oxo-4H-benzo[1,4]oxazin-4-yl)ethoxy]phenyl}-2-ethoxypropanoate

The procedure is as in Preparation 3 with the replacement of the product obtained in Step A of Preparation 1 with the product obtained in Step A of Preparation 7.

### Preparation 11: Ethyl 3-{4-[2-(7-benzoyl-3-oxo-4H-benzo[1,4]oxazin-4-yl)ethoxy]phenyl}-2-(2,2,2-trifluoroethoxy)propanoate

The procedure is as in Preparation 4 with the replacement of the product obtained in Step A of Preparation 1 with the product obtained in Step A of Preparation 7.

### EXAMPLE 1

### 3-{4-[7-(Methoxyiminophenyl)methyl-3-oxo-4H-benzo[1,4]thiazin-4-yl)ethoxy]phenyl}-2-ethoxypropanoate

1 g of the compound obtained in Preparation 1 is dissolved in 30 ml of dimethylformamide and then 0.92 g of potassium carbonate is added and stirring is carried out at 80° C. for one hour. Ethyl 3-[4-(2-chloroethoxy)phenyl]-2-ethoxypropanoate (1.51 g) is dissolved in a minimum amount of dimethylformamide and stirring is carried out overnight at 110° C.

The dimethylformamide is then evaporated off, and the solution is subsequently hydrolysed and acidified. The aqueous phase is extracted with dichloromethane, dried over magnesium sulphate and filtered and the organic phase is evaporated. The residue is purified on silica gel using toluene and ethyl acetate (8/2) as eluant to yield the title product in the form of an oil.

### Elemental Microanalysis:

| % | C | H | N |
|---|---|---|---|
| Calculated: | 66.17 | 6.09 | 4.98 |
| Found: | 65.58 | 6.28 | 4.71 |

### EXAMPLE 2

### Ethyl 3-{4-[7-(methoxyiminophenyl)methyl-3-oxo-4H-benzo[1,4]thiazin-4-yl)ethoxy]phenyl}-2-(2,2,2-trifluoroethoxy)propanoate

1 g of the compound obtained in Preparation 1 is dissolved in 30 ml of dimethylformamide and then 0.91 g of potassium carbonate is added and stirring is carried out at 80° C. for one hour. Ethyl 3-[4-(2-chloroethoxy)phenyl]-2-(2,2,2-trifluoroethoxy)propanoate (1.78 g) is dissolved in a minimum amount of dimethylformamide and stirring is carried out overnight at 110° C.

The dimethylformamide is then evaporated off, and the solution is subsequently hydrolysed and acidified. The aqueous phase is extracted with dichloromethane, dried over magnesium sulphate and filtered and the organic phase is evaporated. The residue is purified on silica gel using toluene and ethyl acetate (8/2) as eluant to yield the title product in the form of a green oil.

### Elemental Microanalysis:

| % | C | H | N |
|---|---|---|---|
| Calculated: | 60.38 | 5.07 | 4.54 |
| Found: | 60.79 | 5.25 | 4.34 |

### EXAMPLE 3

### Ethyl 3-{4-[6-(methoxyiminophenyl)methyl-3-oxo-4H-benzo[1,4]oxazin-4-yl)ethoxy]phenyl}-2-ethoxypropanoate

1 g of the compound obtained in Preparation 2 is dissolved in 30 ml of dimethylformamide and then 1 g of potassium carbonate is added and stirring is carried out at 80° C. for one hour. Ethyl 3-[4-(2-chloroethoxy)phenyl]-2-ethoxypropanoate (1.59 g) is dissolved in a minimum amount of dimethylformamide and stirring is carried out overnight at 110° C.

The dimethylformamide is then evaporated off, and the solution is subsequently hydrolysed and acidified. The aqueous phase is extracted with dichloromethane, dried over magnesium sulphate and filtered and the organic phase is evaporated. The residue is purified on silica gel using toluene and ethyl acetate (8/2) as eluant to yield the title product.

### EXAMPLE 4

### Ethyl 3-{4-[7-(hydroxyiminophenyl)methyl-3-oxo-4H-benzo[1,4]thiazin-4-yl)ethoxy]phenyl}-2-ethoxypropanoate

1 g of the compound obtained in Preparation 3 is dissolved in 20 ml of ethanol and then 0.4 g of hydroxylamine hydrochloride and 0.6 g of pyridine are added. The mixture is heated at reflux for 4 hours with stirring.

The mixture is hydrolysed and then acidified with 3N HCl and the solution is extracted with ethyl acetate, dried, filtered and evaporated. The residue is purified on silica gel using toluene and ethyl acetate (8/2) as eluant to yield the title product in the form of an oil.

### Elemental Microanalysis:

| % | C | H | N |
|---|---|---|---|
| Calculated: | 65.67 | 5.88 | 5.11 |
| Found: | 65.85 | 6.27 | 5.63 |

### EXAMPLE 5

### Ethyl 3-{4-[7-(hydroxyiminophenyl)methyl-3-oxo-4H-benzo[1,4]thiazin-4-yl)ethoxy]phenyl}-2-(2,2,2-trifluoroethoxy)propanoate

1 g of the compound obtained in Preparation 4 is dissolved in 20 ml of ethanol and then 0.36 g of hydroxylamine hydrochloride and 0.5 g of pyridine are added. The mixture is heated at reflux for 4 hours with stirring.

The mixture is hydrolysed and then acidified with 3N HCl and the solution is extracted with ethyl acetate, dried, filtered and evaporated. The residue is purified on silica gel using toluene and ethyl acetate (8/2) as eluant to yield the title product in the form of a greenish powder.

### Elemental Microanalysis:

| % | C | H | N |
|---|---|---|---|
| Calculated: | 59.79 | 4.85 | 4.65 |
| Found: | 60.08 | 5.13 | 4.28 |

### EXAMPLE 6

### Ethyl 3-{4-[6-(hydroxyiminophenyl)methyl-3-oxo-4H-benzo[1,4]oxazin-4-yl)ethoxy]phenyl}-2-ethoxypropanoate

1 g of the compound obtained in Preparation 5 is dissolved in 20 ml of ethanol and then 0.41 g of hydroxylamine hydrochloride and 0.6 g of pyridine are added. The mixture is heated at reflux for 4 hours with stirring.

The mixture is hydrolysed and then acidified with 3N HCl and the solution is extracted with ethyl acetate, dried, filtered and evaporated. The residue is purified on silica gel using toluene and ethyl acetate (8/2) as eluant to yield the title product in the form of an oil.

### EXAMPLE 7

### 3-{4-[7-(Hydroxyiminophenyl)methyl-3-oxo-4H-benzo[1,4]thiazin-4-yl)ethoxy]phenyl}-2-ethoxypropanoic acid

1 g of the compound obtained in Example 4 is dissolved in a tetrahydrofuran/water mixture (12/8 ml), and then 0.13 g of lithium hydroxide dissolved in a minimum amount of water is added. The reaction mixture is stirred overnight at 50° C.

The tetrahydrofuran is then evaporated off and then the solution is hydrolysed and subsequently acidified with 3N HCl. Extraction with ethyl acetate, drying, filtration and evaporation of the organic phase are then carried out. The residue is purified on silica gel using dichloromethane and methanol (95/5) as eluant to yield the title product in the form of a beige powder.

Melting point: 74-76° C.

### Elemental Microanalysis:

| % | C | H | N |
|---|---|---|---|
| Calculated: | 64.60 | 5.42 | 5.38 |
| Found: | 64.32 | 5.89 | 5.02 |

### EXAMPLE 8

### 3-{4-[7-(Methoxyiminophenyl)methyl-3-oxo-4H-benzo[1,4]thiazin-4-yl)ethoxy]phenyl}-2-ethoxypropanoic acid

1 g of the compound obtained in Example 1 is dissolved in a tetrahydrofuran/water mixture (12/8 ml), and then 0.13 g of lithium hydroxide dissolved in a minimum amount of water is added. The reaction mixture is stirred overnight at 50° C.

The tetrahydrofuran is then evaporated off and then the solution is hydrolysed and subsequently acidified with 3N HCl. Extraction with ethyl acetate, drying, filtration and evaporation of the organic phase are then carried out. The residue is purified on silica gel using dichloromethane and methanol (95/5) as eluant to yield the title product in the form of a yellowish powder.

Melting point: 77-79° C.

### Elemental Microanalysis:

| % | C | H | N |
|---|---|---|---|
| Calculated: | 65.15 | 5.66 | 5.23 |
| Found: | 65.33 | 5.90 | 5.57 |

### EXAMPLE 9

### 3-{4-[7-(Hydroxyiminophenyl)methyl-3-oxo-4H-benzo[1,4]thiazin-4-yl)ethoxy]phenyl}-2-(2,2,2-trifluoroethoxy)propanoic acid

1 g of the compound obtained in Example 5 is dissolved in a tetrahydrofuran/water mixture (12/8 ml), and then 0.12 g of lithium hydroxide dissolved in a minimum amount of water is added. The reaction mixture is stirred overnight at 50° C.

The tetrahydrofuran is then evaporated off and then the solution is hydrolysed and subsequently acidified with 3N HCl. Extraction with ethyl acetate, drying, filtration and evaporation of the organic phase are then carried out. The residue is purified on silica gel using dichloromethane and methanol (95/5) as eluant to yield the title product in the form of a green powder.

Melting point: 85-88° C.

### Elemental Microanalysis:

| % | C | H | N |
|---|---|---|---|
| Calculated: | 58.53 | 4.39 | 4.88 |
| Found: | 58.39 | 4.68 | 4.42 |

### EXAMPLE 10

### 3-{4-[7-(Methoxyiminophenyl)methyl-3-oxo-4H-benzo[1,4]thiazin-4-yl)ethoxy]phenyl}-2-(2,2,2-trifluoroethoxy)propanoic acid

1 g of the compound obtained in Example 2 is dissolved in a tetrahydrofuran/water mixture (12/8 ml), and then 0.11 g of lithium hydroxide dissolved in a minimum amount of water is added. The reaction mixture is stirred overnight at 50° C.

The tetrahydrofuran is then evaporated off and then the solution is hydrolysed and subsequently acidified with 3N HCl. Extraction with ethyl acetate, drying, filtration and evaporation of the organic phase are then carried out. The residue is purified on silica gel using dichloromethane and methanol (95/5) as eluant to yield the title product in the form of a green powder.

Melting point: 64-66° C.

### Elemental Microanalysis:

| % | C | H | N |
|---|---|---|---|
| Calculated: | 59.18 | 4.62 | 4.76 |
| Found: | 59.53 | 4.31 | 4.54 |

### EXAMPLE 11

### 3-{4-[6-(Hydroxyiminophenyl)methyl-3-oxo-4H-benzo[1,4]oxazin-3-yl)ethoxy]phenyl}-2-ethoxypropanoic acid

1 g of the compound obtained in Example 6 is dissolved in a tetrahydrofuran/water mixture (12/8 ml), and then 0.13 g of lithium hydroxide dissolved in a minimum amount of water is added. The reaction mixture is stirred overnight at 50° C.

The tetrahydrofuran is then evaporated off and then the solution is hydrolysed and subsequently acidified with 3N HCl. Extraction with ethyl acetate, drying, filtration and evaporation of the organic phase are then carried out. The residue is purified on silica gel using dichloromethane and methanol (95/5) as eluant to yield the title product in the form of a beige powder.

Melting point: 75-76° C.

### Elemental Microanalysis:

| % | C | H | N |
|---|---|---|---|
| Calculated: | 66.66 | 5.59 | 5.55 |
| Found: | 66.53 | 5.31 | 5.34 |

### EXAMPLE 12

### 3-{4-[6-(Methoxyiminophenyl)methyl-3-oxo-4H-benzo[1,4]oxazin-4-yl)ethoxy]phenyl}-2-ethoxypropanoic acid

1 g of the compound obtained in Example 3 is dissolved in a tetrahydrofuran/water mixture (12/8 ml), and then 0.13 g of lithium hydroxide dissolved in a minimum amount of water is added. The reaction mixture is stirred overnight at 50° C.

The tetrahydrofuran is then evaporated off and then the solution is hydrolysed and subsequently acidified with 3N HCl. Extraction with ethyl acetate, drying, filtration and evaporation of the organic phase are then carried out. The residue is purified on silica gel using dichloromethane and methanol (95/5) as eluant to yield the title product in the form of a yellowish powder.

Melting point: 63-65° C.

### Elemental Microanalysis:

| % | C | H | N |
|---|---|---|---|
| Calculated: | 67.17 | 5.83 | 5.40 |
| Found: | 67.53 | 6.11 | 5.34 |

### EXAMPLE 13

### Ethyl 3-{4-[6-(methoxyiminophenyl)methyl-3-oxo-4H-benzo[1,4]thiazin-4-yl)ethoxy]phenyl}-2-ethoxypropanoate

The procedure is as in Example 1 with replacement of the compound of Preparation 1 with the compound of Preparation 6. The title product is obtained in the form of an oil.

### Elemental Microanalysis:

| % | C | H | N |
|---|---|---|---|
| Calculated: | 66.17 | 6.09 | 4.98 |
| Found: | 66.53 | 6.31 | 4.94 |

### EXAMPLE 14

### Ethyl 3-{4-[6-(methoxyiminophenyl)methyl-3-oxo-4H-benzo[1,4]thiazin-4-yl)ethoxy]phenyl}-2-(2,2,2-trifluoroethoxy)propanoate

The procedure is as in Example 2 with replacement of the compound of Preparation 1 with the compound of Preparation 6. The title product is obtained in the form of a yellow oil.

### Elemental Microanalysis:

| % | C | H | N |
|---|---|---|---|
| Calculated: | 60.38 | 5.07 | 4.54 |
| Found: | 60.69 | 5.25 | 4.34 |

### EXAMPLE 15

### Ethyl 3-{4-[7-(methoxyiminophenyl)methyl-3-oxo-4H-benzo[1,4]oxazin-4-yl)ethoxy]phenyl}-2-ethoxypropanoate

The procedure is as in Example 1 with replacement of the compound of Preparation 1 with the compound of Preparation 7. The title product is obtained in the form of an oil.

### Elemental Microanalysis:

| % | C | H | N |
|---|---|---|---|
| Calculated: | 68.12 | 6.27 | 5.12 |

### EXAMPLE 16

### Ethyl 3-{4-[7-(methoxyiminophenyl)methyl-3-oxo-4H-benzo[1,4]oxazin-4-yl)ethoxy]phenyl}-2-(2,2,2-trifluoroethoxy)propanoate

The procedure is as in Example 2 with replacement of the compound of Preparation 1 with the compound of Preparation 7. The title product is obtained in the form of a colourless oil.

### Elemental Microanalysis:

| % | C | H | N |
|---|---|---|---|
| Calculated: | 62.00 | 5.20 | 4.66 |
| Found: | 61.79 | 5.25 | 4.59 |

### EXAMPLE 17

### Ethyl 3-{4-[6-(hydroxyiminophenyl)methyl-3-oxo-4H-benzo[2,3]thiazin-4-yl)ethoxy]phenyl}-2-ethoxypropanoate

The procedure is as in Example 4 with replacement of the compound of Preparation 3 with the compound of Preparation 8. The title product is obtained in the form of a yellow powder.

Melting point: 65-66° C.

### Elemental Microanalysis:

| % | C | H | N |
|---|---|---|---|
| Calculated: | 65.67 | 5.88 | 5.11 |
| Found: | 65.89 | 5.60 | 5.75 |

### EXAMPLE 18

### Ethyl 3-{4-[6-(hydroxyiminophenyl)methyl-3-oxo-4H-benzo[1,4]thiazin-4-yl)ethoxy]phenyl}-2-(2,2,2-trifluoroethoxy)propanoate

The procedure is as in Example 5 with replacement of the compound of Preparation 4 with the compound of Preparation 9. The title product is obtained in the form of a white powder.

Melting point: 171-173° C.

### Elemental Microanalysis:

| % | C | H | N |
|---|---|---|---|
| Calculated: | 59.79 | 4.85 | 4.65 |
| Found: | 60.01 | 5.07 | 4.38 |

### EXAMPLE 19

### Ethyl 3-{4-[7-(hydroxyiminophenyl)methyl-3-oxo-4H-benzo[1,4]oxazin-4-yl)ethoxy]phenyl}-2-ethoxypropanoate

The procedure is as in Example 4 with replacement of the compound of Preparation 3 with the compound of Preparation 10. The title product is obtained in the form of a yellow powder.

Melting point: 84-86° C.

### Elemental Microanalysis:

| % | C | H | N |
|---|---|---|---|
| Calculated: | 67.66 | 6.06 | 5.26 |
| Found: | 67.33 | 6.41 | 4.90 |

### EXAMPLE 20

### Ethyl 3-{4-[7-(hydroxyiminophenyl)methyl-3-oxo-4H-benzo[1,4]oxazin-4-yl)ethoxy]phenyl}-2-(2,2,2-trifluoroethoxy)propanoate

The procedure is as in Example 5 with replacement of the compound of Preparation 4 with the compound of Preparation 11. The title product is obtained in the form of a white powder.

Melting point: 84-86° C.

### Elemental Microanalysis:

| % | C | H | N |
|---|---|---|---|
| Calculated: | 61.43 | 4.98 | 4.78 |
| Found: | 61.08 | 5.29 | 4.51 |

### EXAMPLE 21

### 3-{4-[6-(Methoxyiminophenyl)methyl-3-oxo-4H-benzo[2,3]thiazin-4-yl)ethoxy]phenyl}-2-ethoxypropanoic acid

The procedure is as in Example 8 with replacement of the compound of Example 1 with the compound of Example 13. The title product is obtained in the form of a yellow powder.

Melting point: 58-61° C.

### Elemental Microanalysis:

| % | C | H | N |
|---|---|---|---|
| Calculated: | 65.03 | 5.83 | 5.23 |
| Found: | 65.13 | 5.61 | 5.34 |

### EXAMPLE 22

### 3-{4-[6-(Methoxyiminophenyl)methyl-3-oxo-4H-benzo[1,4]thiazin-4-yl)ethoxy]phenyl}-2-(2,2,2-trifluoroethoxy)propanoic acid

The procedure is as in Example 10 with replacement of the compound of Example 2 with the compound of Example 14. The title product is obtained in the form of a white powder.

Melting point: 66-68° C.

### Elemental Microanalysis:

| % | C | H | N |
|---|---|---|---|
| Calculated: | 59.18 | 4.62 | 4.76 |
| Found: | 58.79 | 4.71 | 4.62 |

### EXAMPLE 23

### 3-{4-[6-(Hydroxyiminophenyl)methyl-3-oxo-4H-benzo[2,3]thiazin-4-yl)ethoxy]phenyl}-2-ethoxypropanoic acid

The procedure is as in Example 7 with replacement of the compound of Example 4 with the compound of Example 17. The title product is obtained in the form of a beige powder.

Melting point: 127-130° C.

### EXAMPLE 24

### 3-{4-[6-(Hydroxyiminophenyl)methyl-3-oxo-4H-benzo[1,4]thiazin-4-yl)ethoxy]phenyl}-2-(2,2,2-trifluoroethoxy)propanoic acid

The procedure is as in Example 9 with replacement of the compound of Example 5 with the compound of Example 18. The title product is obtained in the form of a white powder.

Melting point: 97-99° C.

### Elemental Microanalysis:

| % | C | H | N |
|---|---|---|---|
| Calculated: | 58.53 | 4.39 | 4.88 |
| Found: | 58.39 | 4.68 | 4.72 |

### EXAMPLE 25

### 3-{4-[7-(Methoxyiminophenyl)methyl-3-oxo-4H-benzo[1,4]oxazin-4-yl)ethoxy]phenyl}-2-ethoxypropanoic acid

The procedure is as in Example 8 with replacement of the compound of Example 1 with the compound of Example 15. The title product is obtained in the form of a white powder.

Melting point: 54-57° C.

### Elemental Microanalysis:

| % | C | H | N |
|---|---|---|---|
| Calculated: | 67.17 | 5.83 | 5.40 |
| Found: | 67.20 | 6.08 | 5.21 |

### EXAMPLE 26

### 3-{4-[7-(Methoxyiminophenyl)methyl-3-oxo-4H-benzo[1,4]oxazin-4-yl)ethoxy]phenyl}-2-(2,2,2-trifluoroethoxy)propanoic acid

The procedure is as in Example 10 with replacement of the compound of Example 2 with the compound of Example 16. The title product is obtained in the form of a yellow powder.

Melting point: 61-64° C.

### Elemental Microanalysis:

| % | C | H | N |
|---|---|---|---|
| Calculated: | 60.84 | 4.75 | 4.89 |
| Found: | 60.53 | 4.41 | 4.64 |

### EXAMPLE 27

### 3-{4-[7-(Hydroxyiminophenyl)methyl-3-oxo-4H-benzo[1,4]oxazin-3-yl)ethoxy]phenyl}-2-ethoxypropanoic acid

The procedure is as in Example 7 with replacement of the compound of Example 4 with the compound of Example 19. The title product is obtained in the form of a white powder.

### Melting point 95-97° C.

### Elemental Microanalysis:

| % | C | H | N |
|---|---|---|---|
| Calculated: | 66.66 | 5.59 | 5.55 |
| Found: | 66.53 | 5.31 | 5.34 |

### EXAMPLE 28

### 3-{4-[7-(Hydroxyiminophenyl)methyl-3-oxo-4H-benzo[1,4]oxazin-4-yl)ethoxy]phenyl}-2-(2,2,2-trifluoroethoxy)propanoic acid

The procedure is as in Example 9 with replacement of the compound of Example 5 with the compound of Example 20. The title product is obtained in the form of a white powder.

Melting point: 86-88° C.

### Elemental Microanalysis:

| % | C | H | N |
|---|---|---|---|
| Calculated: | 60.22 | 4.51 | 5.02 |
| Found: | 59.86 | 4.74 | 4.81 |

### Pharmacological Study

### EXAMPLE A

### Acute Toxicity Study

Acute toxicity was evaluated after oral administration to groups each comprising 8 mice (26±2 grams). The animals were observed at regular intervals during the course of the first day, and daily for two weeks following the treatment. The LD₅₀ (dose that causes the death of 50% of the animals) was evaluated and demonstrated the low toxicity of the compounds of the invention.

### EXAMPLE B

### Effectiveness in Genetic Models

Mutations in laboratory animals and also different sensitivities to dietary regimens have allowed the development of animal models having non-insulin-dependent diabetes and hyperlipidaemia associated with obesity and with resistance to insulin.

Genetic mice models (ob/ob) (Diabetes, 1982, 31 (1), 1-6) and Zucker (fa/fa) rats have been developed by various laboratories in order to understand the physiopathology of those diseases and test the effectiveness of new antidiabetic compounds (Diabetes, 1983, 32, 830-838).

### Antidiabetic and Hypolipaemic Effect in the ob/ob Mouse

The 10-week-old female ob/ob mouse (Harlan) is used for the in vivo tests. The animals are kept in a light-darkness cycle of 12 hours at 25° C. The mouse has a basal hyperglycaemia of 2 g/l. The animals are randomly selected with regard to their glycaemia to form groups of six. The compounds tested by the intraperitoneal route are dissolved in a mixture of dimethyl sulphoxide (10%) and Solutol (15%) to be administered at 10 mg/kg in a volume of 2.5 ml/kg twice per day for four days. By the per os route, the compounds are tested at 30 mg/kg administered in a volume of 2.5 ml/kg of 1% HEC twice per day for four days. The control groups receive the solvents under the same conditions as the treated groups.

The activity of the products is evaluated by measuring glycaemia, triglycerides and insulinaemia 24 hours after the final administration and by measuring body weight daily.

The compounds of the invention demonstrate a very good capacity to lower glycaemia, triglycerides and insulinaemia: by way of example, the compound of Example 11 administered at a dose of 3 mg/kg shows a 25% reduction in glycaemia, a 45% reduction in triglycerides and a 22% reduction in insulinaemia, with an insignificant variation in body weight, while under the same conditions rosiglitazone exhibits a significant increase in four days. In addition, no side effect was observed during the in vivo tests.

### EXAMPLE C

### Pharmaceutical Composition

1000 tablets each containing a dose of 5 mg of 3-{4-[6-(hydroxyiminophenyl)methyl-3-oxo-4H-benzo[1,4]oxazin-3-yl)ethoxy]phenyl}-2-ethoxypropanoic acid (Example 11) 5 g
wheat starch . . . 20 g
maize starch . . . 20 g
lactose . . . 30 g
magnesium stearate . . . 2 g
silica . . . 1 g
hydroxypropyl cellulose . . . 2 g

### EXAMPLE D

### Variation in Body Weight

Male C57 Black 6 ob/ob mice 8 to 12 weeks old were used. After being placed in quarantine for one week, they were weighed and then selected randomly with regard to weight, and 6 homogeneous groups (starting weight not significantly different) were formed. After having been weighed, the different associations to be tested are injected intraperitoneally once per day for 7 days. The molecules are injected in a 5% DMSO/15% Solutol/q.s. H₂O solution heated to 65° C. to ensure good dissolution. The solution is in addition preheated prior to injection. The mice are weighed every day and the weight attained after 7 days of treatment is recorded.

The results obtained clearly demonstrate:
that the association according to the invention between a compound of formula (I) and an antioxidant agent allows a significant reduction in the weight of the obese mice,
that there is a synergy between the 2 components of the association, the loss in weight ascertained being far greater using the association than when using each component administered on its own.

### EXAMPLE E

### Pharmaceutical Composition

100 tablets each containing a dose of 30 mg of 3-{4-[6-(hydroxyiminophenyl)methyl-3-oxo-4H-benzo[1,4]oxazin-3-yl)ethoxy]phenyl}-2-ethoxypropanoic acid and 10 mg of coenzyme Q₁₀

3-{4-[6-(hydroxyiminophenyl)methyl-3-oxo-4H-benzo[1,4]oxazin-3-yl)ethoxy]phenyl}-2-ethoxypropanoic acid (Example 11) . . . 3 g
coenzyme Q₁₀ . . . 1 g
wheat starch . . . 20 g
maize starch . . . 20 g
lactose . . . 30 g
magnesium stearate . . . 2 g
silica . . . 1 g
hydroxypropyl cellulose . . . 2 g

1-28. (canceled)

29. A compound selected from those of formula (I): 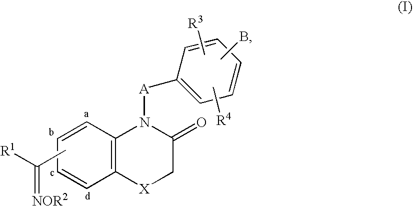
wherein:
X represents an oxygen atom or a sulphur atom,
A represents a (C₁-C₆)alkylene chain in which a CH₂ group may optionally be replaced by a hetero atom selected from oxygen and sulphur, or by an NRₐ group (wherein Rₐ represents a hydrogen atom or a linear or branched (C₁-C₆)alkyl group), or by a phenylene or naphthylene group,
R¹ and R², which may be identical or different, each represents a hydrogen atom, a linear or branched (C₁-C₆)alkyl group, a linear or branched (C₂-C₆)alkenyl group, a linear or branched (C₂-C₆)alkynyl group, an aryl group, an aryl-(C₁-C₆)alkyl group in which the alkyl moiety may be linear or branched, an aryl-(C₂-C₆)alkenyl group in which the alkenyl moiety may be linear or branched, an aryl-(C₂-C₆)alkynyl group in which the alkynyl moiety may be linear or branched, a heteroaryl group, a heteroaryl-(C₁-C₆)alkyl group in which the alkyl moiety may be linear or branched, a heteroaryl-(C₂-C₆)alkenyl group in which the alkenyl moiety may be linear or branched, a heteroaryl-(C₂-C₆)alkynyl group in which the alkynyl moiety may be linear or branched, a (C₃-C₈)cycloalkyl group, a (C₃-C₈)cycloalkyl-(C₁-C₆)alkyl group in which the alkyl moiety may be linear or branched or a linear or branched (C₁-C₆)polyhaloalkyl group,
R³ and R⁴, which may be identical or different, each represents a hydrogen atom, a halogen atom, or an R, OR or NRR′ group wherein R and R′, which may be identical or different, each represents a hydrogen atom or a linear or branched (C₁-C₆)alkyl group, a linear or branched (C₂-C₆)alkenyl group, a linear or branched (C₂-C₆)alkynyl group, an aryl group, an aryl-(C₁-C₆)alkyl group in which the alkyl moiety may be linear or branched, an aryl-(C₂-C₆)alkenyl group in which the alkenyl moiety may be linear or branched, an aryl-(C₂-C₆)alkynyl group in which the alkynyl moiety may be linear or branched, a heteroaryl group, a heteroaryl-(C₁-C₆)alkyl group in which the alkyl moiety may be linear or branched, a heteroaryl-(C₂-C₆)alkenyl group in which the alkenyl moiety may be linear or branched, a heteroaryl-(C₂-C₆)alkynyl group in which the alkynyl moiety may be linear or branched, a (C₃-C₈)cycloalkyl group, a (C₃-C₈)cycloalkyl-(C₁-C₆)alkyl group in which the alkyl moiety may be linear or branched or a linear or branched (C₁-C₆)polyhaloalkyl group,
or R³ and R⁴, together with the carbon atoms carrying them, when they are carried by two adjacent carbon atoms, form a ring that comprises 5 or 6 ring members, wherein the ring may optionally contain a hetero atom selected from oxygen, sulphur and nitrogen,
B represents a linear or branched (C₁-C₆)alkyl group or a linear or branched (C₂-C₆)alkenyl group, those groups being substituted:
by a group of formula (II): 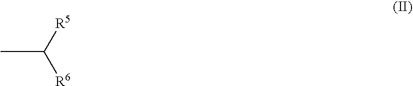
wherein:
R⁵ represents a 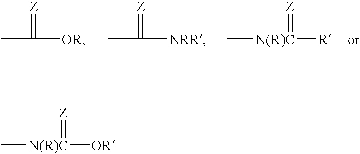
group wherein Z represents an oxygen atom or a sulphur atom,
and R⁶ represents an aryl group, an arylalkyl group in which the alkyl moiety contains from 1 to 6 carbon atoms and may be linear or branched, a heteroaryl group, a heteroarylalkyl group in which the alkyl moiety contains from 1 to 6 carbon atoms and may be linear or branched, CN, tetrazole 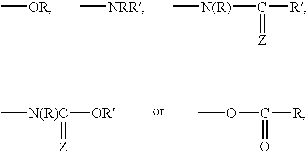
or by a group R⁷, wherein R⁷ represents a CN, tetrazole, 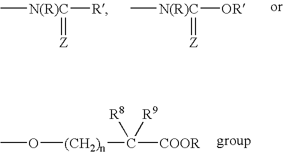
wherein n represents 0, 1, 2, 3, 4, 5 or 6, and R⁸ and R⁹, which may be identical or different, each represents a hydrogen atom or a linear or branched (C₁-C₆)alkyl group, wherein R⁸ and R⁹ cannot simultaneously represent a hydrogen atom,
or B represents a group of formula (II) or a group R⁷,
it being understood that:
the oxime R¹—C(═N—OR²)— may have the Z or E configuration,
aryl means a phenyl, naphthyl or biphenyl group, which groups may optionally be partially hydrogenated,
heteroaryl means an aromatic mono- or bi-cyclic group containing from 5 to 10 ring members, wherein the bicyclic heteroaryl groups may optionally be partially hydrogenated on one of the rings, and wherein each ring contains from 1 to 3 hetero atoms selected from oxygen, nitrogen and sulphur,
wherein the aryl and heteroaryl groups may optionally be substituted by from 1 to 3 identical or different groups selected from linear or branched (C₁-C₆)alkyl, linear or branched (C₁-C₆)polyhaloalkyl, linear or branched (C₁-C₆)alkoxy, hydroxy, carboxy, formyl, linear or branched (C₁-C₆)acyl, aroyl, NR_{b}R_{c} (wherein R_{b} and R_{c}, which may be identical or different, each represents a hydrogen atom, a linear or branched (C₁-C₆)alkyl group, an aryl group or a heteroaryl group), ester, amido, nitro, cyano, and halogen atoms,
its enantiomers and diastereoisomers, and pharmaceutically acceptable addition salts thereof with an acid or a base.

30. The compound of claim 29, wherein the group R¹—C(═N—OR²)— is in the b or c position.

31. The compound of claim 29, wherein R³ and R⁴ represent a hydrogen atom.

32. The compound of claim 29, wherein A represents an ethyleneoxy group.

33. The compound of claim 29, wherein R² represents a hydrogen atom.

34. The compound of claim 29, wherein R² represents an alkyl group.

35. The compound of claim 29, wherein R¹ an unsubstituted or substituted phenyl group.

36. The compound of claim 29, wherein B represents an alkyl group substituted by a group 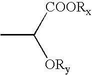
wherein Rₓ and R_{y} which may be identical or different, each represents a hydrogen atom or an alkyl group.

37. The compound of claim 29, wherein B represents a group 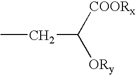
wherein Rₓ and R_{y}, which may be identical or different, each represents a hydrogen atom or an alkyl group.

38. The compound of claim 29, which is selected from:
ethyl 2-ethoxy-3-[4-(2-{7-[(methoxyimino)(phenyl)methyl]-3-oxo-2,3-dihydro-4H-1,4-benzothiazin-4-yl}ethoxy)phenyl]propanoate,
ethyl 3-[4-(2-{7-[(methoxyimino)(phenyl)methyl]-3-oxo-2,3-dihydro-4H-1,4-benzothiazin-4-yl}ethoxy)phenyl]-2-(2,2,2-trifluoroethoxy)propanoate,
ethyl 2-ethoxy-3-[4-(2-{6-[(methoxyimino)(phenyl)methyl]-3-oxo-2,3-dihydro-4H-1,4-benzoxazin-4-yl}ethoxy)phenyl]propanoate,
ethyl 2-ethoxy-3-[4-(2-{7-[(hydroxyimino)(phenyl)methyl]-3-oxo-2,3-dihydro-4H-1,4-benzothiazin-4-yl}ethoxy)phenyl]propanoate,
ethyl 3-[4-(2-{7-[(hydroxyimino)(phenyl)methyl]-3-oxo-2,3-dihydro-4H-1,4-benzothiazin-4-yl}ethoxy)phenyl]-2-(2,2,2-trifluoroethoxy)propanoate,
ethyl 2-ethoxy-3-[4-(2-{6-[(hydroxyimino)(phenyl)methyl]-3-oxo-2,3-dihydro-4H-1,4-benzoxazin-4-yl}ethoxy)phenyl]propanoate,
2-ethoxy-3-[4-(2-{7-[(hydroxyimino)(phenyl)methyl]-3-oxo-2,3-dihydro-4H-1,4-benzothiazin-4-yl}ethoxy)phenyl]propanoic acid,
2-ethoxy-3-[4-(2-{7-[(methoxyimino)(phenyl)methyl]-3-oxo-2,3-dihydro-4H-1,4-benzothiazin-4-yl}ethoxy)phenyl]propanoic acid,
3-[4-(2-{7-[(hydroxyimino)(phenyl)methyl]-3-oxo-2,3-dihydro-4H-1,4-benzothiazin-4-yl}ethoxy)phenyl]-2-(2,2,2-trifluoroethoxy)propanoic acid,
3-[4-(2-{7-[(methoxyimino)(phenyl)methyl]-3-oxo-2,3-dihydro-4H-1,4-benzothiazin-4-yl}ethoxy)phenyl]-2-(2,2,2-trifluoroethoxy)propanoic acid,
2-ethoxy-3-[4-(2-{6-[(hydroxyimino)(phenyl)methyl]-3-oxo-2,3-dihydro-4H-1,4-benzoxazin-4-yl}ethoxy)phenyl]propanoic acid,
2-ethoxy-3-[4-(2-{6-[(methoxyimino)(phenyl)methyl]-3-oxo-2,3-dihydro-4H-1,4-benzoxazin-4-yl}ethoxy)phenyl]propanoic acid,
enantiomers and diastereoisomers thereof, and pharmaceutically acceptable addition salts thereof with an acid or a base.

39. A pharmaceutical composition comprising as active ingredient at least one compound of claim 29, or a pharmaceutically acceptable addition salt thereof with an acid or a base, alone or in combination with one or more pharmaceutically acceptable excipients.

40. A method of treating a living animal body, including a human, afflicted with a condition selected from hyperglycaemia, dyslipidaemia, non-insulin-dependent type II diabetes, insulin resistance, glucose intolerance, disorders associated with syndrome X, coronary artery disease, cardiovascular diseases, renal disorders, retinopathy, disorders associated with the activation of endothelial cells, psoriasis, polycystic ovary syndrome, dementia, osteoporosis, intestinal inflammatory disorders, myotonic dystrophy, pancreatitis, arteriosclerosis, xanthoma, type I diabetes, obesity, conditions requiring regulation of appetite, anorexia, bulimia, anorexia nervosa, cancer, and conditions requiring an angiogenesis inhibitor, comprising the step of administering to the living animal body, including a human, an amount of a compound of claim 29 which is effective for treatment of the condition.

41. The method of claim 40, wherein the cancer is selected from hormone-dependent cancer, breast cancer, and colon cancer.

42. A composition comprising a combination of the compound of claim 29 and an antioxidant agent.

43. The composition of claim 42, wherein the compound of claim 29 is 3-{4-[6-(hydroxyiminophenyl)methyl-3-oxo-4H-benzo[1,4]oxazin-3-yl)ethoxy]phenyl}-2-ethoxypropanoic acid, an enantiomer or diastereoisomer thereof, or a pharmaceutically acceptable addition salts thereof with an acid or a base.

44. The composition of claim 42, wherein the antioxidant agent is coenzyme Q₁₀.

45. The composition of claim 42, wherein the antioxidant agent is vitamin E.

46. The composition of claim 42, which is 3-{4-[6-(hydroxyiminophenyl)methyl-3-oxo-4H-benzo[1,4]oxazin-3-yl)ethoxy]phenyl}-2-ethoxypropanoic acid and coenzyme Q₁₀.

47. The composition of claim 42, further comprising one or more pharmaceutically acceptable excipients.

48. A method of treating a living animal body, including a human, afflicted with obesity, comprising the step of administering to the living animal body, including a human, an amount of a composition of claim 42, which is effective for treatment of obesity.

49. A method of treating a living animal body, including a human, afflicted with obesity caused by a therapeutic treatment, comprising the step of administering to the living animal body, including a human, an amount of a composition of claim 42, which is effective for treatment of obesity caused by a therapeutic treatment.

50. A method of treating a living animal body, including a human, afflicted with obesity caused by treatment of type I or type II diabetes, comprising the step of administering to the living animal body, including a human, an amount of a composition of claim 42, which is effective for treatment of obesity caused by treatment of type I or type II diabetes.

51. A method of treating a living animal body, including a human, afflicted with overweight characterised by a body mass index greater than 25 and less than 30, comprising the step of administering to the living animal body, including a human, an amount of a composition of claim 42, which is effective for treatment of overweight characterised by a body mass index greater than 25 and less than 30.

52. A method of treating a living animal body, including a human, afflicted with overweight characterised by a body mass index greater than 25 and less than 30 caused by a therapeutic treatment, comprising the step of administering to the living animal body, including a human, an amount of a composition of claim 42, which is effective for treatment of overweight characterised by a body mass index greater than 25 and less than 30 caused by a therapeutic treatment.

53. A method of treating a living animal body, including a human, afflicted with overweight characterised by a body mass index greater than 25 and less than 30 caused by treatment of type I or type II diabetes, comprising the step of administering to the living animal body, including a human, an amount of a composition of claim 42, which is effective for treatment of overweight characterised by a body mass index greater than 25 and less than 30 caused by a treatment of type I or type II diabetes.
